(19) United States
(12) Patent Application Publication
(10) Pub. No.: US 2022/0370485 A1
(43) Pub. Date: Nov. 24, 2022

(54) COMPOUNDS AND METHODS FOR TREATING OR PREVENTING CARDIOVASCULAR DISEASES AND CONDITIONS

(71) Applicant: Lexicon Pharmaceuticals, Inc., The Woodlands, TX (US)

(72) Inventor: Deepak L. BHATT, Newton, MA (US)

(21) Appl. No.: 17/574,977

(22) Filed: Jan. 13, 2022

Related U.S. Application Data
(60) Provisional application No. 63/137,323, filed on Jan. 14, 2021.

(51) Int. Cl. A61K 31/7034; A61K 9/00; A61P 13/12; A61P 9/04

(52) U.S. Cl. CPC A61K 31/7034; A61K 9/0053; A61P 13/12; A61P 9/04

(57) ABSTRACT

This invention relates to methods of treating and/or preventing cardiovascular diseases and conditions and to methods of reducing the risk of cardiovascular death, reducing the risk of hospitalization for heart failure, reducing the risk of hospitalization for heart failure, reducing the risk of urgent visits for heart failure, and slowing the progression of kidney disease.

This application claims priority to U.S. provisional patent application No. 63/137,323, filed Jan. 14, 2021.

### 1. FIELD OF THE INVENTION

This invention relates to compounds useful in, and methods of treating and/or preventing cardiovascular diseases and conditions, including managing heart failure (e.g., reducing the risk of cardiovascular death, hospitalization, or urgent visits due to heart failure) and preventing myocardial infarction and stroke.

### 2. BACKGROUND

Multiple isoforms of the sodium-glucose transporter are expressed in the human body. Inhibitors of sodium-glucose cotransporter 2 (SGLT2) were initially developed to treat hyperglycemia in patients with diabetes mellitus. Subsequently, some were shown to lower the risk of hospitalization for heart failure among patients with type 2 diabetes, who are at substantial risk for this complication. See, e.g., Zinman B, Wanner C, Lachin J M, et al., “Empagliflozin, cardiovascular outcomes, and mortality in type 2 diabetes,” N Engl J Med 2015; 373:2117-28. In addition, some SGLT2 inhibitors have been shown to reduce the risk of death from cardiovascular causes or hospitalization for heart failure in patients with heart failure (with or without diabetes) and a reduced ejection fraction. McMurray J J V, Solomon S D, Inzucchi S E, et al., “Dapagliflozin in patients with heart failure and reduced ejection fraction,” N Engl J Med 2019; 381:1995-2008; Packer M, Anker S D, Butler J, et al., “Cardiovascular and renal outcomes with empagliflozin in heart failure” N Engl J Med 2020; 383:1413-24.

However, the safety and potential efficacy of initiating SGLT2 inhibition soon after an episode of decompensated heart failure remain uncertain. Potential safety concerns include the risks of hypotension and precipitation of kidney failure among patients with fluctuating volume status and renal function who are receiving treatment with other drugs that might also affect the glomerular filtration rate (GFR). In addition, whether the benefits of SGLT2 inhibition extend to patients with heart failure with preserved ejection fraction remains unknown.

A complicating factor is that many diabetes patients suffer from coexisting chronic kidney disease, which itself raises the risk of heart failure and ischemic events. And while some clinical trials have addressed the use of SGLT2 inhibitors in such patients, they required the presence of macroalbuminuria for inclusion, in addition to reduced estimated glomerular filtration rate (eGFR). See, e.g., Udell J A, Bhatt D L, Braunwald E, et al., “Saxagliptin and cardiovascular outcomes in patients with type 2 diabetes and moderate or severe renal impairment: observations from the SAVOR-TIMI 53 Trial,” Diabetes Care 2015; 38:696-705; Neal B, Perkovic V, Mahaffey K W, et al., “Canagliflozin and cardiovascular and renal events in type 2 diabetes,” N Engl J Med 2017; 377:644-57; Heerspink H J L, Stefansson B V, Correa-Rotter R, et al., “Dapagliflozin in patients with chronic kidney disease,” N Engl J Med 2020; 383:1436-46. The efficacy and safety of sodium-glucose cotransporter 2 inhibitors such as sotagliflozin in preventing cardiovascular events in patients with diabetes with chronic kidney disease with or without albuminuria have not been well studied.

Unlike previously tested sodium-glucose cotransporter inhibitors, the compound sotagliflozin is a dual SGLT1/2 inhibitor, i.e., it inhibits both SGLT1 and SGLT2. Because SGLT1 is the dominant isoform of cardiac SGLT (Kashiwagi, Yusuke et al., “Expression of SGLT1 in Human Hearts and Impairment of Cardiac Glucose Uptake by Phlorizin during Ischemia-Reperfusion Injury in Mice,” PLOS ONE 2015; 10(6): e0130605) results of heart failure studies using conventional SGLT2 inhibitors cannot readily be extrapolated to sotagliflozin. In short, while sotagliflozin has been approved in Europe for the treatment of type 1 diabetes, its effectiveness in the treatment of other diseases and disorders remains unknown.

### 3. SUMMARY OF THE INVENTION

This invention encompasses methods of treating and/or preventing cardiovascular diseases and conditions such as myocardial infarction and stroke. It also encompasses methods of reducing the risk of cardiovascular death, reducing the risk of hospitalization for heart failure, reducing the risk of hospitalization for heart failure, and reducing the risk of urgent visits for heart failure in a patient in need thereof. The invention also encompassed methods of managing heart failure (e.g., slowing the progression of or managing one or more symptoms of heart failure) in a patient. It further encompasses a method of slowing or preventing the progression of kidney disease in a patient. Each of the methods encompassed by this invention comprise administering to a patient a prophylactically or therapeutically effective amount of sotagliflozin or a pharmaceutical composition comprising a prophylactically or therapeutically effective amount of sotagliflozin.

Typical patients have type 2 diabetes. In some embodiments of the invention, the patient is an adult. In some, the patient has at least one additional risk factor for heart failure (e.g., worsening heart failure). Examples of additional risk factors for heart failure include high blood pressure, coronary artery disease, a history of heart attack or stroke, myocarditis, congenital heart defect, and arrhythmia. In some embodiments of the invention, the patient has chronic kidney disease (e.g., CKD2, CKD3, or CKD4).

### 4. BRIEF DESCRIPTION OF THE FIGURES

Aspects of this invention may be understood with reference to the attached figures, briefly described below.

FIG. 1. Total Number of Deaths from Cardiovascular Causes, Hospitalizations for Heart Failure, and Urgent Visits for Heart Failure. Shown is a time-to-event analysis of the estimated number of events per 100 patients for the primary end point of the total number of deaths from cardiovascular causes, hospitalizations for heart failure, and urgent visits for heart failure in the sotagliflozin and placebo groups. Total events after randomization are shown as estimated cumulative events per 100 patients instead of events per 100 patient-years to graphically show the time course of event accrual during follow-up. The number of competing deaths not from cardiovascular causes was 91 in the sotagliflozin group and 76 in the placebo group. Dotted lines indicate a 95% confidence interval for events per 100 patients.

FIG. 2. First Occurrence of Death from Cardiovascular Causes, Nonfatal Myocardial Infarction, or Nonfatal Stroke. Shown is a time-to-event analysis of the original primary end point of the estimated cumulative incidence for the first occurrence of death from cardiovascular causes, nonfatal myocardial infarction, or nonfatal stroke in the sotagliflozin and placebo groups. The number of competing deaths not from cardiovascular causes was 80 in the sotagliflozin group and 69 in the placebo group. Dotted lines indicate a 95% confidence interval for the cumulative incidence.

FIG. 3. Primary Efficacy End-Point Events. Shown are the rates of primary efficacy end-point events (deaths from cardiovascular causes and hospitalizations and urgent visits for heart failure) in the sotagliflozin group and the placebo group. Total events after randomization are shown as estimated cumulative events per 100 patients instead of events per 100 patient-years to graphically show the time course of event accrual during follow-up. Competing deaths from non-cardiovascular causes occurred in 14 patients in the sotagliflozin group and in 18 patients in the placebo group.

FIG. 4. Primary Efficacy End-Point Events in Select Prespecified Subgroups. Shown are the hazard ratios with 95% confidence intervals for primary end-point events in select prespecified sub-groups. The confidence intervals have not been adjusted for multiple testing, and inferences drawn from the intervals may not be reproducible. Left ventricular ejection fraction (LVEF) was categorized according to the randomization stratification factor. GFR denotes glomerular filtration rate.

FIG. 5. First Occurrence of Either Death from Cardiovascular Causes or Hospitalization for Heart Failure. Shown is the cumulative incidence of the first occurrence of either death from cardiovascular causes or hospitalization for heart failure in the sotagliflozin group and the placebo group. Competing deaths from non-cardiovascular causes occurred in 10 patients in the sotagliflozin group and in 13 patients in the placebo group.

### 5. DETAILED DESCRIPTION

This invention is based on the results of human clinical trials of the compound sotagliflozin, a dual SGLT1/2 inhibitor chemically named (2S,3R,4R,5S,6R)-2-(4-chloro-3-(4-ethoxybenzyl)phenyl)-6-(methylthio)tetrahydro-2H-pyran-3,4,5-triol and having the structure:

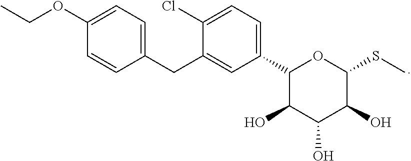

Solid forms of sotagliflozin have been disclosed. See, e.g., U.S. Pat. No. 8,217,156. Oral solid dosage forms of the compound have also been disclosed. See, e.g., U.S. patent application publication no. US-2012-0172320-A1.

### 5.1. Definitions

Unless otherwise indicated, the term “combination” when referred to administration of two or more drugs to a patient means that the two or more drugs are both administered (e.g., self-administered) to the patient so that the patient receives the benefit of the two or more drugs (e.g., during a given day). However, it is not necessary that the two or more drugs be administered at the same time or by the same route of administration.

Unless otherwise indicated, the term “include” has the same meaning as “include, but are not limited to,” and the term “includes” has the same meaning as “includes but is not limited to.” Similarly, the term “such as” has the same meaning as the term “such as, but not limited to.”

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder or changing the way that a patient responds to the disease or disorder.

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition or one or more symptoms associated with the disease or condition, prevent its recurrence, or slow or stop its worsening. A “prophylactically effective amount” of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A “therapeutically effective amount” of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or retards or slows the progression of the disease or disorder.

Unless otherwise indicated, the term “urgent visit” refers to a patient's visit to a heath care provider (e.g., a hospital, clinic or doctor) resulting from an acute cardiovascular event (e.g., heart attack, stroke) or one or more symptoms associated with a cardiovascular event.

### 5.2. Methods of Use and Examples

This invention encompasses methods of reducing the risk of cardiovascular death, reducing the risk of hospitalization for heart failure, reducing the risk of hospitalization for heart failure, and reducing the risk of urgent visits for heart failure in a patient in a patient which comprise administering to the patient a prophylactically effective amount of sotagliflozin. The invention also encompassed methods of managing heart failure (e.g., slowing the progression of or managing one or more symptoms of heart failure) in a patient.

Typical patients have type 2 diabetes. In some embodiments, the patient is an adult. In some, the patient has at least one additional risk factor for heart failure (e.g., worsening heart failure). Examples of additional risk factors for heart failure include high blood pressure, coronary artery disease, a history of heart attack or stroke, myocarditis, congenital heart defect, and arrhythmia.

In some embodiments of the invention, the patient has chronic kidney disease (e.g., CKD2, CKD3, or CKD4). In some embodiments, the patient has an eGFR of less than 30 ml/min/173 m². In others, the patient has eGFR of 30 to <45 ml/min/173 m². In others, the patient has an eGFR of ≥45 ml/min/173 m². In some embodiments of the invention, the patient has albuminuria.

In some embodiments of the invention, the patient has an ejection fraction of <40%. In some, the patient has an ejection fraction of 40% to <50%. In some, the patient has an ejection fraction of ≥50%.

In some methods of the invention, the sotagliflozin is administered orally (e.g., in a tablet or capsule) to the patient. Typically, the prophylactically effective amount of sotagliflozin is at least 200 mg per day (e.g., 200, 250, 300, 350, or 400 mg/day).

In some methods of the invention, the sotagliflozin is administered in combination with a RAAS inhibitor, beta blocker, or diuretic (e.g., loop diuretic), or glucose-lowering medication. Examples of RAAS inhibitors include is an ACE inhibitor, an angiotensin-receptor blocker, an angiotensin receptor-neprilysin inhibitor, or a mineralocorticoid-receptor antagonist. Examples of glucose-lowering medication include metformin, a sulfonylurea, a DPP-4 inhibitor (e.g., sitagliptin), insulin, or a GLP-1 receptor agonist.

5.2.1. Study of Diabetic Patients with Chronic Kidney Disease

A multicenter, double-blind clinical trial was conducted in which patients with type 2 diabetes mellitus (glycated hemoglobin level, ≥7%), chronic kidney disease (estimated glomerular filtration rate, 25 to 60 ml per minute per 1.73 m² of body-surface area), and risks for cardiovascular disease were randomly assigned in a 1:1 ratio to receive sotagliflozin or placebo. The primary end point was changed during the trial to the composite of the total number of deaths from cardiovascular causes, hospitalizations for heart failure, and urgent visits for heart failure.

Of 19,188 patients screened, 10,584 were enrolled, with 5,292 assigned to the sotagliflozin group and 5,292 assigned to the placebo group and followed for a median of 16 months. The rate of primary end-point events was 5.6 events per 100 patient-years in the sotagliflozin group and 7.5 events per 100 patient-years in the placebo group (hazard ratio, 0.74; 95% confidence interval [CI], 0.63 to 0.88; P<0.001). The rate of deaths from cardiovascular causes per 100 patient-years was 2.2 with sotagliflozin and 2.4 with placebo (hazard ratio, 0.90; 95% CI, 0.73 to 1.12; P=0.35). For the original coprimary end point of the first occurrence of death from cardiovascular causes, nonfatal myocardial infarction, or nonfatal stroke, the hazard ratio was 0.84 (95% CI, 0.72 to 0.99); for the original coprimary end point of the first occurrence of death from cardiovascular causes or hospitalization for heart failure, the hazard ratio was 0.77 (95% CI, 0.66 to 0.91). Diarrhea, genital mycotic infections, volume depletion, and diabetic ketoacidosis were more common with sotagliflozin than with placebo.

It was found that in patients with diabetes and chronic kidney disease, with or without albuminuria, sotagliflozin resulted in a lower risk of the composite of deaths from cardiovascular causes, hospitalizations for heart failure, and urgent visits for heart failure than placebo.

5.2.1.1. Trial Design

This was a phase 3, randomized, double-blind, placebo-controlled trial that compared sotagliflozin (200 mg once daily, with an increase to 400 mg once daily if unacceptable side effects did not occur) with placebo in patients with type 2 diabetes mellitus, chronic kidney disease, and additional cardiovascular risk; all the patients also received standard-of-care treatments. Randomization was stratified according to criteria for heart failure (ejection fraction of ≤40% documented within the past year or hospitalization for heart failure during the previous 2 years) and geographic region (North America, Latin America, western Europe, eastern Europe, or rest of the world).

Patients

Persons 18 years of age or older with type 2 diabetes mellitus with a glycated hemoglobin level of 7% or higher, chronic kidney disease (eGFR, 25 to 60 ml per minute per 1.73 m2 of body-surface area), and additional cardiovascular risk factors were enrolled. The risk factors consisted of at least one major cardiovascular risk factor in those 18 years of age or older or at least two minor cardiovascular risk factors in those 55 years of age or older. One exclusion criterion was any plan to start an SGLT2 inhibitor during the trial. Written informed consent was obtained from all the patients.

End Points

The original coprimary end points, assessed in time-to-event analyses, were the first occurrence of a major adverse cardiovascular event (MACE, defined as death from cardiovascular causes, non-fatal myocardial infarction, or nonfatal stroke) and the first occurrence of death from cardiovascular causes or hospitalization for heart failure. The primary end point was the total number of deaths from cardiovascular causes, hospitalizations for heart failure, and urgent visits for heart failure.

The secondary end points were the total number of hospitalizations for heart failure and urgent visits for heart failure; deaths from cardiovascular causes; the total number of deaths from cardiovascular causes, hospitalizations for heart failure, nonfatal myocardial infarctions, and nonfatal strokes; the total number of deaths from cardiovascular causes, hospitalizations for heart failure, urgent visits for heart failure, and events of heart failure during hospitalization; the first occurrence of the composite of a sustained decrease of at least 50% in the eGFR from baseline for at least 30 days, long-term dialysis, renal trans-plantation, or a sustained eGFR of less than 15 ml per minute per 1.73 m² for at least 30 days; deaths from any cause; and the total number of deaths from cardiovascular causes, nonfatal myocardial infarctions, and nonfatal strokes. The use of total events allowed for a single patient to have more than one event contributing to the analysis. Subgroup analyses were prespecified.

Statistical Analysis

All efficacy analyses followed the intention-to-treat principle. To allow for analyses of total events, competing-risk marginal models for recurrent events that were stratified according to heart-failure criteria and geographic region, with deaths not included in a given end point treated as competing terminal events, were applied to generate hazard ratios with Wald 95% confidence intervals and P values. Andersen P K, Angst J, Ravn H., “Modeling marginal features in studies of recurrent events in the presence of a terminal event,” Lifetime Data Anal 2019; 25:681-95. Proportionality was confirmed by interaction terms between trial-group assignment and the logarithm of time. Event rates were summarized by the number of events per 100 patient-years of follow-up (Stukel T A, Glynn R J, Fisher E S, Sharp S M, Lu-Yao G, Wennberg J E, “Standardized rates of recurrent outcomes,” Stat Med 1994; 13:1781-91), and accrual of events over time was summarized by cumulative incidence functions. For subgroup analyses, 95% confidence intervals are reported without adjustment for multiple comparisons, and no conclusions can be drawn from these data.

Total events of myocardial infarction and total events of stroke were evaluated post hoc. Testing for differences between the two groups in adverse events was performed post hoc; P values were obtained from Pearson chi-square tests. Change in the eGFR, glycated hemoglobin level, systolic blood pressure, diastolic blood pressure, and weight over time was analyzed post hoc by means of repeated-measures mixed-effects models with absolute change from baseline as the outcome, a random effect for intercept, and fixed effects for trial-group assignment, baseline value, and time. Changes in these outcomes were also jointly modeled with death from any cause to account for competing risk. Data for patients who discontinued the trial were censored for time-to-event end points, and sensitivity analyses were performed, including imputation of the occurrence of events on the date that the patient was last known to be alive and imputation of the same for the sotagliflozin group only.

5.2.1.2. Results

Patient Characteristics

A total of 19,188 patients were screened, of whom 10,584 were enrolled; 5,292 were assigned to each trial group. The median age of the patients was 69 years; 44.9% were female, and 82.7% were White. Vital status was available for 99.4% of the patients; 142 (1.3%) did not complete final trial visits, of whom 67 had unknown end-of-trial vital status. The median duration of exposure to sotagliflozin was 14.2 months (interquartile range, 10.3 to 18.9), and the median duration of exposure to placebo was 14.3 months (interquartile range, 10.3 to 18.9). The median duration of follow-up was 16.0 months (interquartile range, 12.0 to 20.3) in the sotagliflozin group and 15.9 months (interquartile range, 11.9 to 20.3) in the placebo group. In the sotagliflozin group, 3944 patients (74.5%) had an increase of the dose from 200 to 400 mg; in the placebo group, 4002 (75.6%) had the dose ostensibly increased. Early discontinuation of the trial regimen for reasons other than death or early trial termination occurred in 578 patients (10.9%) in the sotagliflozin group and 597 patients (11.3%) in the placebo group.

Baseline characteristics were similar in the two groups (Table 1).

TABLE 1
Baseline Characteristics of Patients*

| | Sotagliflozin | | Placebo | |
|---|---|---|---|---|
| Characteristic | (N = 5292) | | (N = 5292) | |
| Median age (IQR) - yr | 69 | (63-74) | 69 | (63-74) |
| Female sex - no. (%) | 2347 | (44.3) | 2407 | (45.5) |
| Race or ethnic group - no. (%)† | | | | |
| White | 4402 | (83.2) | 4347 | (82.1) |
| Black | 176 | (3.3) | 188 | (3.6) |
| Asian | 317 | (6.0) | 365 | (6.9) |
| American Indian or Alaska Native | 206 | (3.9) | 216 | (4.1) |
| Native Hawaiian or other Pacific Islander | 25 | (0.5) | 15 | (0.3) |
| Multiple | 109 | (2.1) | 95 | (1.8) |
| Unknown | 57 | (1.1) | 66 | (1.2) |
| Median glycated hemoglobin (IQR) - % | 8.3 | (7.6-9.3) | 8.3 | (7.6-9.4) |
| Median body-mass index (IQR) | 31.9 | (28.1-36.2) | 31.7 | (28.0-36.1) |
| Estimated glomerular filtration rate | | | | |
| Median (IQR) - ml/min/1.73 m2 | 44.4 | (37.0-51.3) | 44.7 | (37.0-51.5) |
| Distribution - no. (%) | | | | |
| <30 ml/min/1.73 m2 | 419 | (7.9) | 394 | (7.4) |
| 30 to <45 ml/min/1.73 m2 | 2347 | (44.3) | 2308 | (43.6) |
| ≥45 ml/min/1.73 m2 | 2526 | (47.7) | 2590 | (48.9) |
| Geographic Region - no. (%) | | | | |
| Eastern Europe | 1613 | (30.5) | 1613 | (30.50 |
| Western Europe | 711 | (13.4) | 709 | (13.4) |
| Latin America | 1586 | (30.0) | 1586 | (30.0) |
| North America | 746 | (14.1) | 747 | (14.1) |
| Rest of the World | 636 | (12.0) | 637 | (12.0) |
| Ejection Fraction of ≤40% within past year or | 1054 | (19.9 | 1054 | (19.9) |
| hospitalization for heart failure during previous 2 | | | | |
| years - no. (%) | | | | |
| History of heart failure - no. (%)‡ | 1640 | (31.0) | 1643 | (31.0) |
| Ejection fraction of <40% | 505 | (9.5) | 528 | (10.0) |
| Ejection fraction of 40 to <50% | 290 | (5.5) | 291 | (5.5) |
| Ejection fraction of ≥50% | 843 | (15.9) | 824 | (15.6) |
| Ejection fraction unknown | 2 | (<0.1) | 0 | |
| Cardiovascular risk factors - no. (%) | | | | |
| At least one major | 4682 | (88.5) | 4699 | (88.8) |
| No major, at least two minor | 405 | (7.7) | 413 | (7.8) |
| No major and less than two minor | 205 | (3.9) | 180 | (3.4) |
| Previous myocardial infarction - no. (%) | 1051 | (19.9) | 1057 | (22.1) |
| Previous coronary revascularization - no. (%) | 1208 | (22.8) | 1167 | (22.1) |
| Previous stroke - no. (%) | 472 | (8.9) | 474 | (9.0) |
| Urinary albumin-to-creatinine ratio§ | | | | |
| Median (IQR) | 74 | (18-486) | 75 | (17-477) |
| Distribution - no. (%) | | | | |
| <30 | 1864 | (35.2) | 1845 | (34.9) |
| 30 to <300 | 1770 | (33.4) | 1819 | (34.4) |
| ≥300 | 1658 | (31.3) | 1628 | (30.8) |
| Median left ventricular ejection fraction (IQR) - %‡ | 60 | (51-64) | 60 | (51-65) |
| Median NT-proBNP (IQR) - pg/ml | 196.0 | (75.1-564.6) | 198.1 | (74.6-560.7) |
| Median systolic blood pressure (IQR) - mm Hg | 138 | (127-149) | 139 | (127-149) |
| Median diastolic blood pressure (IQR) - mm Hg | 78 | (70-85) | 78 | (70-85) |
| Any RAAS inhibitor - no. (%)¶ | 4705 | (88.9) | 4660 | (88.1) |
| ACE inhibitor | 2009 | (38.0) | 2039 | (38.5) |
| Angiotensin-receptor blocker | 2619 | (49.5) | 2562 | (48.4) |
| Angiotensin receptor-neprilysin inhibitor | 66 | (1.2) | 65 | (1.2) |
| Mineralocorticoid-receptor antagonist | 810 | (15.3) | 776 | (14.7) |
| Beta-blocker - no. (%) | 3310 | (62.5) | 3306 | (62.5) |
| Calcium-channel blocker - no. (%) | 2228 | (42.1) | 2202 | (41.6) |
| Loop diuretic - no. (%) | 1869 | (35.3) | 1867 | (35.3) |
| Other diuretic - no. (%) | 1568 | (29.6) | 1605 | (30.3) |
| Any glucose-lowering medication - no. (%) | 5111 | (96.6) | 5136 | (97.1) |
| Metformin | 2907 | (54.9) | 2955 | (55.8) |
| Sulfonylurea | 1400 | (26.5) | 1486 | (28.1) |
| DPP-4 Inhibitor | 1041 | (19.7) | 1044 | (19.7) |
| Insulin | 3389 | (64.0) | 3333 | (63.0) |
| GLP-1 receptor agonist | 310 | (5.9) | 323 | (6.1) |

*Percentages may not total 100 because of rounding. ACE denotes angiotensin-converting enzyme, DPP-4 dipeptidyl peptidase 4, GLP-1 glucagon-like peptide 1, IQR interquartile range, and NT-proBNP N-terminal pro-brain natriuretic peptide.
†Race and ethnic group were reported by the investigators.
‡Ejection fraction was measured within 1 year before screening or during the screening period.
§The ratio was calculated with albumin measured in milligrams and creatinine measured in grams.
¶Some patients were taking more than one renin-angiotensin-aldosterone system (RAAS) inhibitor.

Of all randomly assigned patients, 19.9% had an ejection fraction of 40% or less within the past year or hospitalization for heart failure during the previous two years, and the median left ventricular ejection fraction was 60% (interquartile range 51 to 65). The median glycated hemoglobin level was 8.3%, median body-mass index (the weight in kilograms divided by the square of the height in meters) was 31.8, median eGFR was 44.5 ml per minute per 1.73 m² (interquartile range, 37.0 to 51.4), and median urinary albumin-to-creatinine ratio was 74 (interquartile range, 17 to 481).

End Points

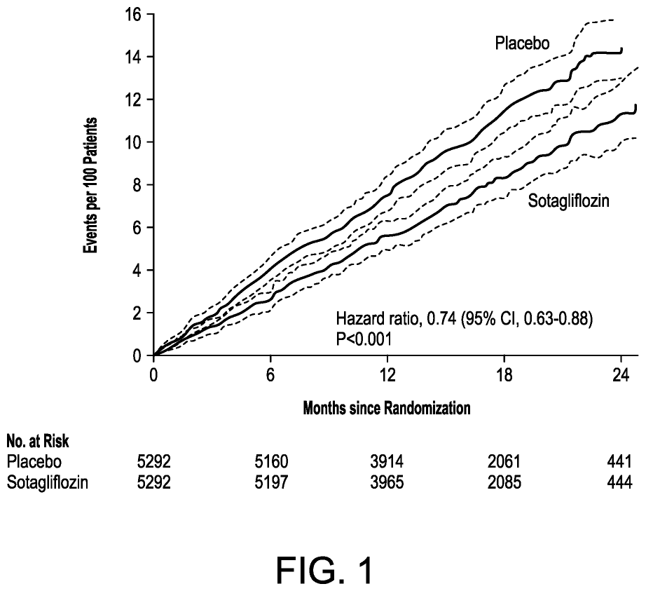

There were 701 first and 930 total primary endpoint events. The rates of total primary end-point events were 5.6 and 7.5 events per 100 patient-years in the sotagliflozin and placebo groups, respectively (hazard ratio, 0.74; 95% confidence interval [CI], 0.63 to 0.88; P<0.001). The estimated time-to-event curves for total primary end-point events are shown in FIG. 1.

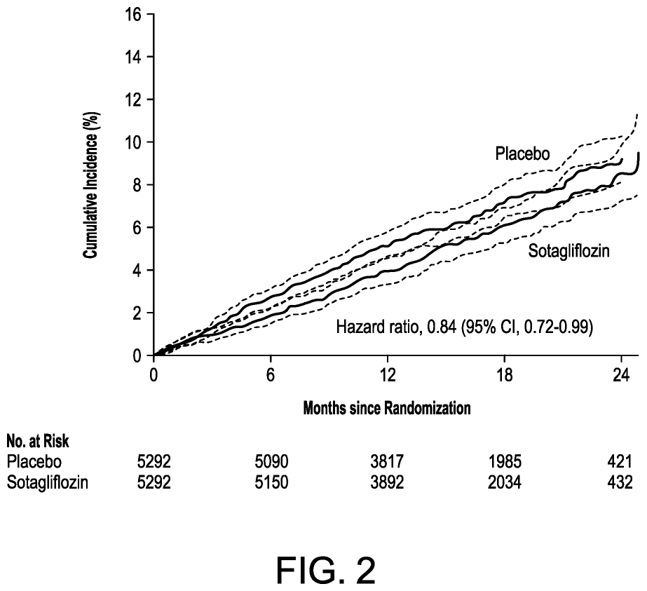

For the total number of hospitalizations for heart failure and urgent visits for heart failure (first secondary end point), the rate was 3.5 events per 100-patient years in the sotagliflozin group and 5.1 events per 100-patient years in the placebo group (hazard ratio, 0.67; 95% CI, 0.55 to 0.82; P<0.001) (Table 2). There was no significant difference between the sotagliflozin and placebo groups in deaths from cardiovascular causes (second secondary end point) (2.2 and 2.4 per 100 patient-years, respectively; hazard ratio, 0.90; 95% CI, 0.73 to 1.12), and the hierarchical analysis stopped at this point. However, results for the composite renal end point did not differ significantly between the two groups, nor did all-cause mortality. The original coprimary end point of the first event of death from cardiovascular causes or hospitalization for heart failure showed a hazard ratio of 0.77 (95% CI, 0.66 to 0.91). The original coprimary end point of the first event of death from cardiovascular causes, nonfatal myocardial infarction, or nonfatal stroke showed a hazard ratio of 0.84 (95% CI, 0.72 to 0.99) (FIG. 2). For investigator-reported events submitted for adjudication before loss of trial funding, 337 of 501 events (67.3%) in the sotagliflozin group and 460 of 664 events (69.3%) in the placebo group were confirmed on adjudication.

TABLE 2
Primary End Point and Secondary End Points.*

| | no. of events/100 | | | |
|---|---|---|---|---|
| | patient-yr (no. of events) | | | |
| | Sotagliflozin | Placebo | Hazard Ratio | |
| End Point | (N = 5292) | (N = 5292) | (95% CI) † | P Value |
| Primary end point: total no. of deaths from | 5.6 (400) | 7.5 (530) | 0.74 (0.63-0.88) | <0.001 |
| cardiovascular causes, hospitalizations | | | | |
| for HF, and urgent visits for HF | | | | |
| Major secondary end points, in order of | | | | |
| hierarchical testing | | | | |
| Total no. of hospitalizations for HF and | 3.5 (245) | 5.1 (360) | 0.67 (0.55-0.82) | <0.001 |
| urgent visits for HF | | | | |
| Deaths from cardiovascular causes | 2.2 (155) | 2.4 (170) | 0.90 (0.73-1.12) | 0.35‡ |
| Total no. of deaths from cardiovascular | 7.6 (541) | 10.4 (738) | 0.72 (0.63-0.83) | — |
| causes, hospitalizations for HF, | | | | |
| nonfatal myocardial infarctions, and | | | | |
| nonfatal strokes | | | | |
| Total no. of deaths from cardiovascular | 6.4 (453) | 8.3 (589) | 0.76 (0.64-0.89) | — |
| causes, hospitalizations for HF, urgent | | | | |
| visits for HF, and events of HF during | | | | |
| hospitalization | | | | |
| First occurrence of a sustained | 0.5 (37) | 0.7 (52) | 0.71 (0.46-1.08) | — |
| decrease of ≥50% in the eGFR from | | | | |
| baseline for ≥30 days, long-term | | | | |
| dialysis, renal transplantation, or | | | | |
| sustained eGFR of <15 ml/min/1.73 | | | | |
| m2 for ≥30 days | | | | |
| Deaths from any cause | 3.5 (246) | 3.5 (246) | 0.99 (0.83-1.18) | — |
| Total no. of deaths from cardiovascular | 4.8 (343) | 6.3 (442) | 0.77 (0.65-0.91) | — |
| causes, nonfatal myocardial | | | | |
| infarctions, and nonfatal strokes | | | | |

*The term eGFR denotes estimated glomerular filtration rate, and HF heart failure.
† Results are presented as point estimates and 95% confidence intervals unadjusted for multiple comparisons, from which no definite conclusions regarding significant differences can be made.
‡The hierarchical analysis stops after the first P value indicating nonsignificance.

Adverse Events

There were no significant differences between the two groups in the percentage of patients with adverse events that occurred or worsened during the treatment period or with events leading to withdrawal of the trial regimen. The percentage of patients with a serious adverse event was 23.4% in the sotagliflozin group and 25.2% in the placebo group. Adverse events of special interest that were more common with sotagliflozin than with placebo were diarrhea (8.5% vs. 6.0%; P<0.001), diabetic ketoacidosis (0.6% vs. 0.3%; P=0.02), genital mycotic infections (2.4% vs. 0.9%; P<0.001), and volume depletion (5.3% vs. 4.0%; P=0.003). There were no significant between-group differences in bone fractures, urinary tract infections, severe hypoglycemia, acute kidney injury (2.2% in the sotagliflozin group and 2.1% in the placebo group; P=0.55), or amputations. The percentage of patients in whom hypertension developed was lower with sotagliflozin than with placebo (2.6% vs. 4.1%), whereas the percentage in whom hypotension developed was higher with sotagliflozin than with placebo (2.6% vs. 1.9%; P=0.009).

Discussion

In this trial, the SGLT1/2 inhibitor sotagliflozin resulted in a lower risk of the composite primary end point of the total number of deaths from cardiovascular causes, hospitalizations for heart failure, and urgent visits for heart failure than placebo (5.6 vs. 7.5 events per 100 patient-years). Deaths from cardiovascular causes and renal end points did not differ significantly between the trial groups. Diarrhea, genital mycotic infections, volume depletion, and diabetic ketoacidosis occurred with sotagliflozin.

This trial did not require that patients have a urinary albumin-to-creatinine ratio of at least 200 or 300, and the trial examined cardiovascular events in a population with a median albumin-to-creatinine ratio of 74. This trial also did not require a history of heart failure or reduced ejection fraction at baseline. Despite the low eGFR in the trial population (median, 44.5 ml per minute per 1.73 m2), kidney injury did not differ significantly between the sotagliflozin and placebo groups.

Potential mechanisms of action of SGLT2 inhibition include renal and systemic natriuretic effects, enhanced myocardial energetics, adaptive cellular reprogramming, increased red-cell mass affecting improved oxygen supply, weight loss, reductions in blood pressure and left ventricular hypertrophy, decreases in uric acid, and beneficial effects on endothelial progenitor cells. See, e.g., Hess D A, Terenzi D C, Trac J Z, et al., “SGLT2 inhibition with empagliflozin increases circulating provascular progenitor cells in people with type 2 diabetes mellitus,” Cell Metab 2019; 30:609-13. Sotagliflozin provides some degree of SGLT1 inhibition as well, slowing intestinal glucose ab-sorption and reducing postprandial glycemia. Mendelian randomization data have raised the possibility that SGLT1 inhibition might be associated with decreased rates of cardiovascular events. Seidelmann S B, Feofanova E, Yu B, et al., “Genetic variants in SGLT1, glucose tolerance, and cardiometabolic risk,” J Am Coll Cardiol 2018; 72:1763-73. The SGLT1 blockade may have contributed to increased reports of diarrhea in our trial.

Investigator-defined end-point events were used for all analyses. Approximately 31% of adjudicated heart failure hospitalizations or urgent visits were not confirmed to be primary events, leading to overestimation of the number of events. See, e.g., Jatene T, Harrington R A, Stone G W, et al., “Investigator-reported bleeding versus post hoc adjudication of bleeding: lessons from the CHAMPION PHOENIX Trial,” J Am Coll Cardiol 2016; 67:596-8. Furthermore, hospitalization for reasons other than heart failure would remove patients from the risk of a primary end-point event, although fewer total hospitalizations in the sotagliflozin group than in the placebo group (1923 vs. 2094) spanning fewer total patient-years (104 vs. 119) suggest the absence of bias favoring sotagliflozin with respect to the primary end point.

The SGLT1/2 inhibitor sotagliflozin resulted in a lower risk of the composite of deaths from cardiovascular causes, hospitalizations for heart failure, and urgent visits for heart failure than placebo among patients with diabetes mellitus and chronic kidney disease, with or without albuminuria. The results in the sotagliflozin group did not differ significantly from those in the placebo group with respect to rates of death from cardiovascular causes or renal end points.

5.2.2. Study of Diabetic Patients with Worsening Heart Failure

A multicenter, double-blind trial in which patients with type 2 diabetes mellitus who were recently hospitalized for worsening heart failure were randomly assigned to receive sotagliflozin or placebo. The primary end point was the total number of deaths from cardiovascular causes and hospitalizations and urgent visits for heart failure (first and subsequent events).

A total of 1,222 patients underwent randomization (608 to the sotagliflozin group and 614 to the placebo group) and were followed for a median of 9.0 months; the first dose of sotagliflozin or placebo was administered before discharge in 48.8% and a median of 2 days after discharge in 51.2%. Among these patients, 600 primary end-point events occurred (245 in the sotagliflozin group and 355 in the placebo group). The rate (the number of events per 100 patient-years) of primary end-point events was lower in the sotagliflozin group than in the placebo group (51.0 vs. 76.3; hazard ratio, 0.67; 95% confidence interval [CI], 0.52 to 0.85; P<0.001). The rate of death from cardiovascular causes was 10.6 in the sotagliflozin group and 12.5 in the placebo group (hazard ratio, 0.84; 95% CI, 0.58 to 1.22); the rate of death from any cause was 13.5 in the sotagliflozin group and 16.3 in the placebo group (hazard ratio, 0.82; 95% CI, 0.59 to 1.14). Diarrhea was more common with sotagliflozin than with placebo (6.1% vs. 3.4%), as was severe hypoglycemia (1.5% vs. 0.3%). The percentage of patients with hypotension was similar in the sotagliflozin group and the placebo group (6.0% and 4.6%, respectively), as was the percentage with acute kidney injury (4.1% and 4.4%, respectively). The benefits of sotagliflozin were consistent in the prespecified subgroups of patients stratified according to the timing of the first dose.

In patients with diabetes and recent worsening heart failure, sotagliflozin therapy, initiated before or shortly after discharge, resulted in a significantly lower total number of deaths from cardiovascular causes and hospitalizations and urgent visits for heart failure than placebo.

5.2.2.1. Trial Design

The trial was a phase 3, double-blind, randomized, placebo-controlled trial. The executive and steering committees (both consisting of academic physicians) and representatives from the sponsors developed the protocol and statistical analysis plan and were responsible for the conduct and oversight of the trial and for the interpretation of the data. The sponsors were responsible for management and monitoring of the trial sites, regulatory reporting, and collection and management of the data. The protocol was approved by the relevant health authority, institutional review board, or ethics committee at each participating site. An independent data and safety monitoring board oversaw the trial.

Eligibility

Patients were eligible for enrollment in the trial if they were 18 to 85 years of age and had been hospitalized because of the presence of signs and symptoms of heart failure and received treatment with intravenous diuretic therapy. Patients were also required to have received a previous diagnosis of type 2 diabetes before the index admission or to have laboratory evidence to support a diagnosis of type 2 diabetes during the index admission. Exclusion criteria included end-stage heart failure or recent acute coronary syndrome, stroke, percutaneous coronary intervention or coronary-artery bypass surgery, or an estimated GFR of less than 30 ml per minute per 1.73 m² of body-surface area. Written informed consent was obtained from all patients.

Patients were excluded if they did not meet criteria for clinical stability before randomization. These criteria included no need for oxygen therapy, a systolic blood pressure of at least 100 mm Hg, no need for intravenous inotropic or vasodilator therapy (excluding nitrates) and having transitioned from intravenous to oral diuretic therapy. Patients were also required to have elevated natriuretic peptide levels at the time of randomization. Natriuretic peptide thresholds were, for B-type natriuretic peptide, at least 150 pg per milliliter (≥450 pg per milliliter for patients with atrial fibrillation) or, for N-terminal pro-B-type natriuretic peptide (NT-proBNP), at least 600 μg per milliliter (≥1800 μg per milliliter for patients with atrial fibrillation).

Trial Procedures

Patients who met all eligibility and stability criteria were randomly assigned, either before or within 3 days after hospital discharge, to receive 200 mg of sotagliflozin once daily (with a dose increase to 400 mg, depending on side effects) or placebo. Randomization was performed centrally with the use of interactive-response technology and was stratified according to left ventricular ejection fraction (<50% or ≥50%) and geographic region of enrollment (North America, Latin America, western Europe, eastern Europe, or rest of the world) at baseline. Follow-up visits were scheduled at 1, 2, and 4 weeks, at 4 months, and every 4 months thereafter.

End Points

The primary end point was the total number of deaths from cardiovascular causes and hospitalizations and urgent visits for heart failure (first and subsequent). The secondary end points were the total number of hospitalizations and urgent visits for heart failure; the incidence of death from cardiovascular causes; the incidence of death from any cause; the total number of deaths from cardiovascular causes, hospitalizations for heart failure, nonfatal myocardial infarctions, and non-fatal strokes; the total number of deaths from cardiovascular causes, hospitalizations and urgent visits for heart failure, and events of heart failure during hospitalization; the change in score on the Kansas City Cardiomyopathy Questionnaire-12 item (KCCQ-12; scores range from 0 to 100, with higher scores indicating better quality of life) to month 4; and the change in the estimated GFR. See, Green C P, Porter C B, Bresnahan D R, Spertus J A., “Development and evaluation of the Kansas City Cardiomyopathy Questionnaire: a new health status measure for heart failure,” J Am Coll Cardiol 2000; 35:1245-55. Adverse events of special interest that occurred during the treatment period were also recorded.

Statistical Analysis

All efficacy analyses were performed according to the intention-to-treat principle. To allow for analyses of the total number of events, competing-risks marginal models for recurrent events (stratified according to left ventricular ejection fraction at baseline [<50% or ≥50%] and geographic region of enrollment [North America, Latin America, western Europe, eastern Europe, or rest of the world]), in which deaths that were not part of a given end point were treated as competing terminal events, were applied to generate hazard ratios (sotagliflozin vs. placebo) with Wald 95% confidence intervals and P values. See, Andersen P K, Angst J, Ravn H., “Modeling marginal features in studies of recurrent events in the presence of a terminal event,” Lifetime Data Anal 2019; 25:681-95. The robust sandwich variance estimate was used for the estimated standard error of the log hazard ratio to account for the dependence of event times within individual patients. See, Lin D Y, Wei Li., “The robust inference for the Cox proportional hazards model,” J Am Stat Assoc 1989; 84:1074-8. Event rates were calculated as the number of events per 100 patient-years of follow-up, and the accrual of events over time was estimated with the use of cumulative incidence functions.

A fixed hierarchical procedure was used to control for type I error in the analyses of the secondary end points. For tertiary and subgroup analyses, 95% confidence intervals were reported without adjustment for multiple testing, and inferences drawn from the intervals may not be reproducible. Change in the KCCQ-12 score from baseline to month four was assessed with the use of analysis of covariance, with trial group as a factor and baseline score and randomization stratification factors as covariates. Change in the estimated GFR over time was analyzed with the use of a repeated-measures, mixed-effects model, with ab-solute change in the estimated GFR from baseline as the outcome, the intercept as a random effect, and trial group, baseline value, and time as fixed effects. A sensitivity analysis of the change in KCCQ-12 score was also performed with the use of a mixed-effects model similar to that used for change in the estimated GFR. In addition, change in the KCCQ-12 score and change in the estimated GFR were jointly modeled with death from any cause to account for competing risk.

5.2.2.2. Results

Patient Characteristics

A total of 1,549 patients underwent screening, of whom 1222 (78.9%) were randomly assigned to a trial group (608 to the sotagliflozin group and 614 to the placebo group) at 306 sites in 32 countries.

The baseline characteristics of the patients are provided in Table 3. The median age of the patients was 70 years, 33.7% were female, and 93.2% were White. A total of 79.1% of the patients had a left ventricular ejection fraction of less than 50%, the median estimated GFR was 49.7 ml per minute per 1.73 m², the median glycated hemoglobin level was 7.1%, and the median NT-proBNP level was 1799.7 pg per milliliter. The first dose of sotagliflozin or placebo was administered before discharge in 48.8% of the patients and after discharge in 51.2% (median, 2 days [interquartile range, 1 to 3] after discharge in both trial groups). The patients were well treated for heart failure with various classes of evidence-based medications, and 85.4% were receiving a glucose-lowering medication.

TABLE 3
Demographic and Clinical Characteristics of the Patients at Baseline.*

| | Sotagliflozin | | Placebo | |
|---|---|---|---|---|
| Characteristic | (N = 608) | | (N = 614) | |
| Median age (IQR) - yr | 69 | (63-76) | 70 | (64-76) |
| Female sex - no. (%) | 198 | (32.6) | 214 | (34.9) |
| Race or ethnic group - no. (%)† | | | | |
| White | 567 | (93.3) | 572 | (93.2) |
| Black | 25 | (4.1) | 25 | (4.1) |
| Asian | 8 | (1.3) | 7 | (1.1) |
| Other | 2 | (0.3) | 4 | (0.7) |
| Unknown | 6 | (1.0) | 6 | (1.0) |
| Median glycated hemoglobin (IQR) - % | 7.1 | (6.4-8.3) | 7.2 | (6.4-8.2) |
| Median body-mass index (IQR)‡ | 30.4 | (26.3-34.3) | 31.1 | (27.3-34.5) |
| Median KCCQ-12 score (IQR)§ | 35 | (28-44) | 35 | (26-44) |
| Median estimated GFR (IQR) - ml/min/1.73 m² | 49.2 | (39.5-61.2) | 50.5 | (40.5-64.6) |
| Geographic Region - no. (%)¶ | | | | |
| Eastern Europe | 244 | (40.1) | 246 | (40.1) |
| Western Europe | 155 | (25.5) | 155 | (25.2) |
| Latin America | 132 | (21.7) | 134 | (21.8) |
| North America | 39 | (6.4) | 41 | (6.7) |
| Rest of the World | 38 | (6.2) | 38 | (6.2) |
| Diagnosis of diabetes- no. (%) | | | | |
| During the index admission | 17 | (2.8) | 14 | (2.3) |
| Within 3 mo before randomization | 25 | (4.1) | 20 | (3.3) |
| Within 6 mo before randomization | 35 | (5.8) | 34 | (5.5) |
| Left ventricular ejection fraction | | | | |
| Median value (IQR) - % | 35 | (28-47) | 35 | (28-45) |
| <50%-no. (%)¶ | 481 | (79.1) | 485 | (79.0) |
| Median NT-proBNT (IQR) - pg/ml | 1816.8 | (854.7-3658.5) | 1741.0 | (842.5-3582.2) |
| Median blood pressure | | | | |
| Systolic | 122 | (111-135) | 122 | (112-133) |
| Diastolic | 72 | (66-80) | 73 | (66-80) |
| Any RAAS inhibitor - no. (%) | 553 | (91.0) | 563 | (91.7) |
| ACE inhibitor | 254 | (41.8) | 241 | (39.3) |
| ARB | 245 | (40.3) | 270 | (44.0) |
| ARNI | 93 | (15.3) | 112 | (18.2) |
| MRA | 403 | (66.3) | 385 | 962.7) |
| Beta-blocker - no. (%) | 564 | (92.8) | 561 | (91.4) |
| Loop diuretic - no. (%) | 580 | (95.4) | 581 | (94.6) |
| Other diuretic - no. (%) | 66 | (10.9) | 62 | (10.1) |
| Any glucose-lowering medication - no. (%) | 522 | (85.9) | 522 | (85.0) |
| Metformin | 320 | (52.6) | 320 | (52.1) |
| Sulfonylurea | 114 | (18.8) | 114 | (18.6) |
| DPP-4 Inhibitor | 96 | (15.8) | 102 | (16.6) |
| Insulin | 217 | (35.7) | 217 | (35.3) |
| GLP-1 receptor agonist | 17 | (2.8) | 23 | (3.7) |

*Percentages may not total 100 because of rounding. ACE denotes angiotensin-converting enzyme, ARB angiotensin-receptor blocker, ARNI angiotensin receptor-neprilysin inhibitor, DPP-4 dipeptidyl peptidase 4, GFR glomerular filtration rate, GLP-1 glucagon-like peptide 1, IQR interquartile range, MRA mineralocorticoid receptor antagonist, NT-proBNP N-terminal pro-B-type natriuretic peptide, and RAAS renin-angiotensin-aldosterone system.
†Race and ethnic group were reported by the investigators. The category “other” includes native Hawaiian or other Pacific Islander or multiple races.
‡The body-mass index is the weight in kilograms divided by the square of the height in meters.
§Scores on the Kansas City Cardiomyopathy Questionnaire-12 item (KCCQ-12) range from 0 to 100, with higher scores indicating better quality of life.
¶Geographic region and left ventricular ejection fraction below 50% were categorized according to the respective randomization stratification factors.

Follow-Up

At the time of database lock, vital status was available for 97.1% of the patients; 43 patients (3.5%) did not complete the final trial visit, among whom vital status was unknown at the end of the trial for 35. In the sotagliflozin group, the median duration of follow-up was 9.2 months, the median duration of treatment was 7.8 months, and 81.7% of the patients were exposed to the trial agent for at least 80% of follow-up; the corresponding values in the placebo group were 8.9 months, 7.6 months, and 79.2% of the patients. Early discontinuation of the trial regimen for reasons other than death or early termination of the trial occurred in 79 patients (13.0%) in the sotagliflozin group and in 94 patients (15.3%) in the placebo group.

Efficacy End Points

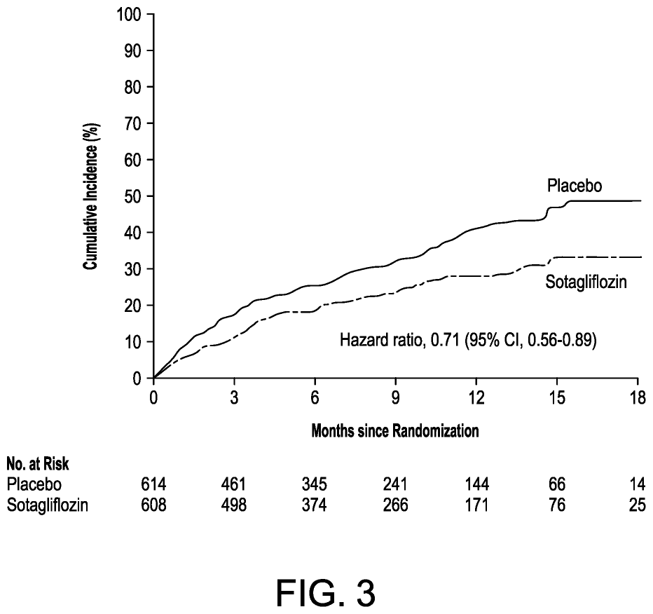

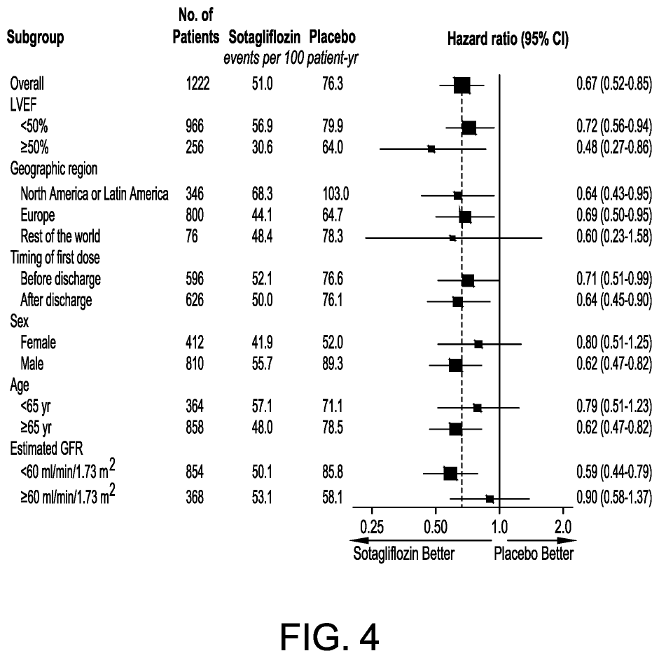

A total of 600 primary end-point events occurred among 1,222 patients (245 in the sotagliflozin group and 355 in the placebo group). The rate of primary end-point events was 51.0 per 100 patient-years in the sotagliflozin group and 76.3 per 100 patient-years in the placebo group (hazard ratio, 0.67; 95% confidence interval [CI], 0.52 to 0.85; P<0.001), for an absolute difference of 25.3 events per 100 patient-years (95% CI, 5.1 to 45.6). See, Stukel T A, Glynn R J, Fisher E S, Sharp S M, Lu-Yao G, Wennberg J E, “Standardized rates of recurrent outcomes,” Stat Med 1994; 13:1781-91. The cumulative incidence curves for the primary efficacy end point are shown in FIG. 3. The results of subgroup analyses of the primary efficacy end point according to stratification factors and select prespecified subgroups showed a consistent treatment effect across the subgroups stratified according to geographic region of enrollment (North America or Latin America, Europe, or rest of the world), left ventricular ejection fraction (<50% or ≥50%), timing of the first dose of sotagliflozin or placebo (before or after discharge), sex, age (<65 years or ≥65 years), and renal function (estimated GFR, <60 ml per minute per 1.73 m² or ≥60 ml per minute per 1.73 m²) (FIG. 4).

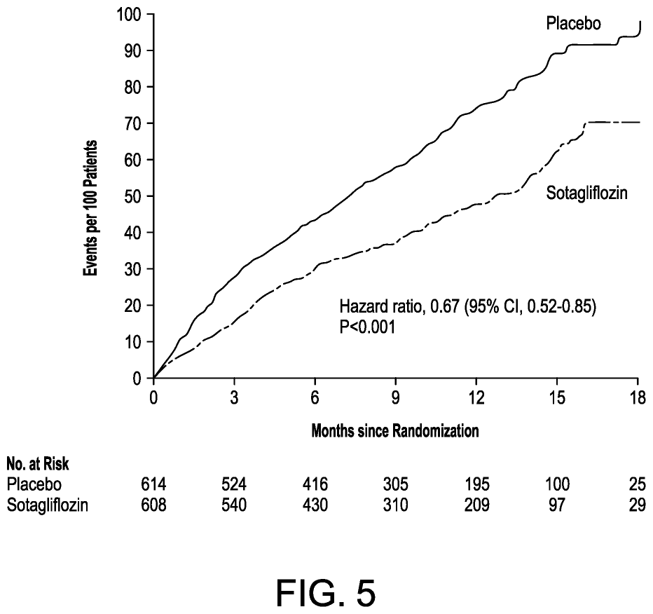

The secondary end points are listed in hierarchical testing order in Table 4. The results of the analysis of the first secondary end point (the total number of hospitalizations and urgent visits for heart failure) were consistent with the results of the primary end-point analysis. The incidence of death from cardiovascular causes or of death from any cause did not differ significantly between the trial groups. To address the possible concern that the primary end point might be subject to double counting of urgent visits for heart failure leading to hospitalization, the total number of deaths from cardiovascular causes and hospitalizations for heart failure, excluding urgent visits for heart failure, was examined. The results were consistent with those of the primary end-point analysis (hazard ratio, 0.68; 95% CI, 0.53 to 0.88). In addition, in a time-to-event analysis of the original primary end point of the trial (the first occurrence of either death from cardiovascular causes or hospitalization for heart failure), the results were consistent with those of the revised primary end point, with a hazard ratio for death from cardiovascular causes or hospitalization for heart failure of 0.71 (95% CI, 0.56 to 0.89) (FIG. 5). The between-group difference in the change in the KCCQ-12 score was 4.1 points (95% CI, 1.3 to 7.0) in favor of the sotagliflozin group, and the between-group difference in the change in the estimated GFR during follow-up was −0.16 ml per minute per 1.73 m² (95% CI, −1.30 to 0.98) in favor of the placebo group.

TABLE 4
Primary End Point and Secondary End Points.

| | | | | | Hazard Ratio or | |
|---|---|---|---|---|---|---|
| | Sotagliflozin | | Placebo | | Difference | |
| End Point | (N = 608) | | (N = 614) | | (95% CI) * | P Value |
| Primary end point: deaths from | 245 | (51.0) | 355 | (76.3) | 0.67 (0.52 to 0.85) | <0.001 |
| cardiovascular causes and | | | | | | |
| hospitalizations and urgent visits | | | | | | |
| for heart failure - total no. of | | | | | | |
| events (rate)† | | | | | | |
| Secondary end points in order of | | | | | | |
| hierarchical testing | | | | | | |
| Hospitalizations and urgent visits | 194 | (40.4) | 297 | (63.9) | 0.64 (0.49 to 0.83) | <0.001 |
| for heart failure - total no. of | | | | | | |
| events (rate)† | | | | | | |
| Deaths from cardiovascular causes - | 51 | (10.6) | 58 | (12.5) | 0.84 (0.58 to 1.22) | 0.36‡ |
| total no. of events (rate)† | | | | | | |
| Deaths from cardiovascular causes, | 247 | (51.4) | 330 | (71.0) | 0.72 (0.56 to 0.92) | — |
| hospitalizations for heart failure, | | | | | | |
| nonfatal myocardial infarctions, and | | | | | | |
| nonfatal strokes - total no. of | | | | | | |
| events (rate)† | | | | | | |
| Deaths from cardiovascular causes, | 263 | (54.7) | 375 | (80.6) | 0.68 (0.54 to 0.86) | — |
| hospitalizationsand urgent visits | | | | | | |
| for heart failure, and events of | | | | | | |
| heart failure during hospitalization - | | | | | | |
| total no. of events (rate)† | | | | | | |
| Deaths from any cause - total no. | 65 | (13.5) | 76 | (16.3) | 0.82 (0.59 to 1.14) | — |
| of events (rate)† | | | | | | |

| | | | | |
|---|---|---|---|---|
| Least-squares mean change in | 17.7 | 13.6 | 4.1 (1.3 to 7.0) | — |
| KCCQ-12 score to month 4 | | | | |
| Least-squares mean change in | −0.34 | −0.18 | −0.16 (−1.30 to 0.98) | — |
| estimated GFR - ml/min/1.73 m² | | | | |

* Hazard ratios (sotagliflozin vs. placebo) are shown for all end points except change in KCCQ-12 score to month 4 and change in estimated GFR, for which differences in the least-squares mean values are shown (sotagliflozin minus placebo).
†Rate was calculated as the number of events per 100 person-years of follow-up.
‡The hierarchical analysis was stopped after the first P value indicating nonsignificance.

The results of sensitivity analyses that used a joint model of the change in the KCCQ-12 score and death from any cause, a joint model of the change in the estimated GFR and death from any cause, and a mixed model of the change in the KCCQ-12 score were similar to those of the primary analysis. Among the investigator-reported events that were submitted for adjudication (before the loss of sponsor funding), 174 of 225 (77.3%) in the sotagliflozin group and 221 of 286 (77.3%) in the placebo group were confirmed on adjudication.

Safety End Points

Serious adverse events that led to withdrawal of sotagliflozin or placebo occurred in 3.0% of the patients in the sotagliflozin group and in 2.8% of the patients in the placebo group. The most common adverse events other than heart failure that occurred in the sotagliflozin group and the placebo group were hypotension (6.0% vs. 4.6%), urinary tract infection (4.8% vs. 5.1%), and diarrhea (6.1% vs. 3.4%). Acute kidney injury occurred in 4.1% of the patients in the sotagliflozin group and in 4.4% of patients in the placebo group. Severe hypoglycemia was more common with sotagliflozin than with placebo (1.5% vs. 0.3%).

Discussion

This clinical trial showed that among patients with diabetes who had worsening heart failure, the primary end point of the total number of cardiovascular deaths and hospitalizations and urgent visits for heart failure was significantly lower with the SGLT1/2 inhibitor sotagliflozin than with placebo. This finding was consistent across multiple prespecified subgroups, including those stratified according to the timing of the first dose of sotagliflozin or placebo (before or after discharge) and left ventricular ejection fraction (reduced or mid-range [<50%] or preserved [≥50%]).

Accumulating evidence from randomized clinical trials supports the use of SGLT2 inhibitors in patients who have stable heart failure (with or without diabetes) and a reduced ejection fraction. See, e.g., Damman K, Beusekamp J C, Boorsma E M, et al., “Randomized, double-blind, placebo-controlled, multicentre pilot study on the effects of empagliflozin on clinical outcomes in patients with acute decompensated heart failure (EMPA-RESPONSE-AHF)” Eur J Heart Fail 2020; 22:713-22. The current trial showed that initiation of a dual SGLT1/2 inhibitor before or shortly after discharge in patients who were hospitalized for worsening heart failure is beneficial. Despite the low estimated GFR (median, 49.7 ml per minute per 1.73 m²) and the recent hospitalization for worsening heart failure in this population, the percentage of patients who had hypotension was similar in the sotagliflozin group and the placebo group, although severe hypoglycemia was more common in the sotagliflozin group. Early initiation of therapy represents an important opportunity to improve outcomes, as indicated by the high rate of primary end-point events at 90 days after randomization among the patients receiving placebo.

In this trial involving patients with diabetes and a recent episode of acute decompensated heart failure, sotagliflozin therapy—whether initiated before or shortly after hospital discharge—resulted in a lower total number of deaths from cardiovascular causes and hospitalizations and urgent visits for heart failure than placebo.

All publications (e.g., patents and patent applications) cited above are incorporated herein by reference in their entireties.

What is claimed is:

1. A method of reducing the risk of cardiovascular death in a patient at risk of cardiovascular death, which comprises administering to the patient a prophylactically effective amount of sotagliflozin.

2. A method of reducing the risk of hospitalization for heart failure in a patient at risk of heart failure, which comprises administering to the patient a prophylactically effective amount of sotagliflozin.

3. A method of reducing the risk of urgent visits for heart failure in a patient at risk of heart failure, which comprises administering to the patient a prophylactically effective amount of sotagliflozin.

4. A method of managing heart failure (e.g., slowing the progression of or managing one or more symptoms of heart failure) in a patient at risk of heart failure, which comprises administering to the patient a prophylactically effective amount of sotagliflozin.

5. A method of reducing the risk of stroke in a patient at risk of suffering a stroke, which comprises administering to the patient a prophylactically effective amount of sotagliflozin.

6. A method of slowing or preventing the progression of kidney disease in a patient with kidney disease or at risk of having kidney disease, which comprises administering to the patient a prophylactically effective amount of sotagliflozin.

7. A method of any of the preceding claims, wherein the patient has type 2 diabetes.

8. A method of any of the preceding claims, wherein the patient has worsening heart failure or at least one additional risk factor for heart failure.

9. The method of claim 8, wherein the additional risk factor for heart failure is high blood pressure, coronary artery disease, a history of heart attack or stroke, myocarditis, congenital heart defect, or arrhythmia.

10. A method of any of claims 1-8, wherein the patient has chronic kidney disease (e.g., CKD2, CKD3, or CKD4).

11. A method of any of claims 1-8, wherein the patient has an ejection fraction of <40%.

12. A method of any of claims 1-8, wherein the patient has an ejection fraction of 40% to <50%.

13. A method of any of claims 1-8, wherein the patient has an ejection fraction of ≥50%.

14. The method of claim 10, wherein the patient has albuminuria.

15. The method of claim 10, wherein the patient has an eGFR of less than 30 ml/min/173 m².

16. The method of claim 10, wherein the patient has an eGFR of 30 to <45 ml/min/173 m².

17. The method of claim 10, wherein the patient has an eGFR of ≥45 ml/min/173 m².

18. A method of any of the preceding claims, wherein sotagliflozin is administered orally.

19. A method of any of the preceding claims, wherein the prophylactically effective amount is at least 200 mg per day (e.g., 200, 250, 300, 350, or 400 mg/day).

20. A method of any of the preceding claims, wherein the sotagliflozin is administered in combination with a RAAS inhibitor, beta blocker, diuretic (e.g., loop diuretic), or glucose-lowering medication.

21. The method of claim 20, wherein the RAAS inhibitor is an ACE inhibitor, an angiotensin-receptor blocker, an angiotensin receptor-neprilysin inhibitor, or a mineralocorticoid-receptor antagonist.

22. The method of claim 20, wherein the glucose-lowering medication is metformin, a sulfonylurea, a DPP-4 inhibitor (e.g., sitagliptin), insulin, or a GLP-1 receptor agonist.
